(19) United States
(12) Patent Application Publication
(10) Pub. No.: US 2024/0325721 A1
(43) Pub. Date: Oct. 3, 2024

(54) PERCUTANEOUS CIRCULATORY SUPPORT DEVICE INCLUDING ANGLED PRESSURE SENSOR

(71) Applicant: Boston Scientific Scimed, Inc., Maple Grove, MN (US)

(72) Inventor: Darrin Dale Beekman, Osseo, MN (US)

(73) Assignee: Boston Scientific Scimed, Inc., Maple Grove, MN (US)

(21) Appl. No.: 18/618,554

(22) Filed: Mar. 27, 2024

Related U.S. Application Data
(60) Provisional application No. 63/455,936, filed on Mar. 30, 2023.

(51) Int. Cl. A61M 60/816; A61M 60/13; A61M 60/165; A61M 60/216; A61M 60/419

(52) U.S. Cl. CPC A61M 60/816; A61M 60/13; A61M 60/165; A61M 60/216; A61M 60/419; A61M 2205/3306; A61M 2205/3327; A61M 2205/3331; A61M 2207/10; A61M 2210/125; A61M 2230/30

(57) ABSTRACT

A percutaneous circulatory support device includes a housing having a proximal end portion, a distal end portion, and a longitudinal axis extending between the proximal end portion and the distal end portion. An impeller is disposed within the housing, and the impeller is configured to rotate relative to the housing to cause blood to flow through the housing. A motor is operably coupled to the impeller, and the motor is configured to rotate the impeller relative to the housing. A pressure sensor is coupled to the housing and is disposed at a non-zero angle relative to the longitudinal axis.

### CROSS REFERENCE TO RELATED APPLICATIONS

This application claims the benefit of U.S. Provisional Patent Application Ser. No. 63/455,936, filed Mar. 30, 2023, which is incorporated herein by reference.

### TECHNICAL FIELD

The present disclosure relates to percutaneous circulatory support systems. More specifically, the disclosure relates to percutaneous circulatory support devices that include one or more pressure sensors.

### BACKGROUND

Percutaneous circulatory support devices can provide transient support for up to approximately several weeks in patients with compromised heart function or cardiac output. Some percutaneous circulatory support devices include one or more pressure sensors for measuring intravascular pressures. Measuring these pressures facilitates, for example, (1) detecting unintended device position changes within the heart, and (2) determining cardiac output, which in turn facilitates evaluation of potential treatment changes. However, devices including pressure sensors may have several drawbacks. For example, sensed pressures may be inaccurate due to dynamic pressure effects. Accordingly, there is a need for improved devices that include pressure sensors.

### SUMMARY

In an Example 1, a percutaneous circulatory support device includes a housing including a proximal end portion, a distal end portion, and a longitudinal axis extending between the proximal end portion and the distal end portion. An impeller is disposed within the housing, and the impeller is configured to rotate relative to the housing to cause blood to flow through the housing. A motor is operably coupled to the impeller, and the motor is configured to rotate the impeller relative to the housing. A pressure sensor is coupled to the housing and is disposed at a non-zero angle relative to the longitudinal axis.

In an Example 2, the percutaneous circulatory support device of Example 1, wherein the housing further includes an internal chamber and an aperture coupled to the internal chamber, and the pressure sensor is disposed within the internal chamber.

In an Example 3, the percutaneous circulatory support device of Example 2, wherein the aperture is a transversely-facing aperture.

In an Example 4, the percutaneous circulatory support device of any of Examples 2-3, further including a sensor mount disposed within the internal chamber and coupled to the pressure sensor.

In an Example 5, the percutaneous circulatory support device of any of Examples 1-4, wherein the housing further includes a plurality of outlet apertures defining a blood outlet and a plurality of struts disposed between the plurality of outlet apertures, and the pressure sensor is carried by one of the plurality of struts.

In an Example 6, the percutaneous circulatory support device of any of Examples 1-5, wherein the pressure sensor includes one of an optical pressure sensor and an electrical pressure sensor.

In an Example 7, the percutaneous circulatory support device of any of Examples 1-6, wherein a direction perpendicular to a sensing membrane of the pressure sensor is disposed at the non-zero angle relative to the longitudinal axis.

In an Example 8, a percutaneous circulatory support device includes a housing having an inlet, a plurality of outlet apertures, and a plurality of struts disposed between the plurality of outlet apertures. An impeller is disposed within the housing, and the impeller is configured to rotate relative to the housing to cause blood to flow into the inlet, through the housing, and out of the plurality of outlet apertures. A motor is operably coupled to the impeller, and the motor is configured to rotate the impeller relative to the housing. A pressure sensor is carried by one of the plurality of struts.

In an Example 9, the percutaneous circulatory support device of Example 8, wherein the housing further includes an internal chamber and an aperture coupled to the internal chamber, and the pressure sensor is disposed within the internal chamber.

In an Example 10, the percutaneous circulatory support device of Example 9, wherein the aperture is a transversely-facing aperture.

In an Example 11, the percutaneous circulatory support device of any of Examples 9-10, wherein the housing includes a proximally-facing aperture coupled to the internal chamber.

In an Example 12, the percutaneous circulatory support device of Example 11, further including a sensor cable coupled to the pressure sensor and extending through the proximally-facing aperture.

In an Example 13, the percutaneous circulatory support device of any of Examples 11-12, wherein the proximally-facing aperture is disposed proximally relative to the outlet.

In an Example 14, the percutaneous circulatory support device of any of Examples 9-13, further including a sensor mount disposed within the internal chamber and coupled to the pressure sensor.

In an Example 15, the percutaneous circulatory support device of any of Examples 9-14, further including a sensor mount disposed within the internal chamber and coupled to the pressure sensor.

In an Example 16, a percutaneous circulatory support device includes a housing having an inlet, an outlet, a proximal end portion, a distal end portion, and a longitudinal axis extending between the proximal end portion and the distal end portion. An impeller is disposed within the housing, and the impeller configured to rotate relative to the housing to cause blood to flow into the inlet, through the housing, and out of the outlet. A motor is operably coupled to the impeller, and the motor is configured to rotate the impeller relative to the housing. A pressure sensor is coupled to the housing and disposed at a non-zero angle relative to the longitudinal axis.

In an Example 17, the percutaneous circulatory support device of Example 16, wherein the housing further includes an internal chamber and an aperture coupled to the internal chamber, and the pressure sensor is disposed within the internal chamber.

In an Example 18, the percutaneous circulatory support device of Example 17, wherein the aperture is a transversely-facing aperture.

In an Example 19, the percutaneous circulatory support device of Example 17, further including a sensor mount disposed within the internal chamber and coupled to the pressure sensor.

In an Example 20, the percutaneous circulatory support device of Example 17, further including a sensor cable coupled to the pressure sensor.

In an Example 21, the percutaneous circulatory support device of Example 20, wherein the aperture is a transversely-facing aperture, the housing further includes a proximally-facing aperture coupled to the internal chamber, and the sensor cable extends through the proximally-facing aperture.

In an Example 22, the percutaneous circulatory support device of Example 17, wherein the housing further includes a plurality of outlet apertures defining the outlet and a plurality of struts disposed between the plurality of outlet apertures, and the pressure sensor is carried by one of the plurality of struts.

In an Example 23, the percutaneous circulatory support device of Example 17, wherein the pressure sensor includes one of an optical pressure sensor and an electrical pressure sensor.

In an Example 24, the percutaneous circulatory support device of Example 16, wherein a direction perpendicular to a sensing membrane of the pressure sensor is disposed at the non-zero angle relative to the longitudinal axis.

In an Example 25, a percutaneous circulatory support device includes a housing having an inlet, an outlet including a plurality of outlet apertures, a plurality of struts disposed between the plurality of outlet apertures. An impeller is disposed within the housing, and the impeller is configured to rotate relative to the housing to cause blood to flow into the inlet, through the housing, and out of the outlet. A motor is operably coupled to the impeller, and the motor is configured to rotate the impeller relative to the housing. A pressure sensor is carried by one of the plurality of struts.

In an Example 26, the percutaneous circulatory support device of Example 25, wherein the housing further includes an internal chamber and an aperture coupled to the internal chamber, and the pressure sensor is disposed within the internal chamber.

In an Example 27, the percutaneous circulatory support device of Example 26, wherein the aperture is a transversely-facing aperture.

In an Example 28, the percutaneous circulatory support device of Example 27, wherein the housing further includes a proximally-facing aperture coupled to the internal chamber.

In an Example 29, the percutaneous circulatory support device of Example 28, further including a sensor cable coupled to the pressure sensor and extending through the proximally-facing aperture.

In an Example 30, the percutaneous circulatory support device of Example 28, wherein the proximally-facing aperture is disposed proximally relative to the outlet.

In an Example 31, the percutaneous circulatory support device of Example 25, further including a sensor mount disposed within the internal chamber and coupled to the pressure sensor.

In an Example 32, a method of manufacturing a percutaneous circulatory support device includes positioning an impeller within a housing such that the impeller is rotatable relative to the housing; operably coupling a motor to the impeller; and coupling a pressure sensor to the housing such that the pressure sensor is disposed at non-zero angle relative to a longitudinal axis of the housing.

In an Example 33, the method of Example 32, wherein coupling the pressure sensor to the housing includes positioning the pressure in an internal chamber of a sensor housing.

In an Example 34, the method of Example 33, wherein the sensor housing further includes an aperture coupled to the internal chamber.

In an Example 35, the method of Example 34, wherein the aperture is a transversely-facing aperture, the sensor housing further includes a proximally-facing aperture, and coupling the pressure sensor to the housing includes inserting the pressure sensor through the proximally-facing aperture.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a side sectional view of an illustrative percutaneous circulatory support device (also referred to herein, interchangeably, as a “blood pump”), in accordance with embodiments of the subject matter disclosed herein.

FIG. 2 is a detail perspective view of the illustrative percutaneous circulatory support device of FIG. 1.

FIG. 3 is another side sectional view of the illustrative percutaneous circulatory support device of FIG. 1.

FIG. 4 is a detail side sectional view of the illustrative percutaneous circulatory support device along line 4-4 of FIG. 3.

FIG. 5 is a flow diagram of an exemplary method of manufacturing a percutaneous circulatory support device, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

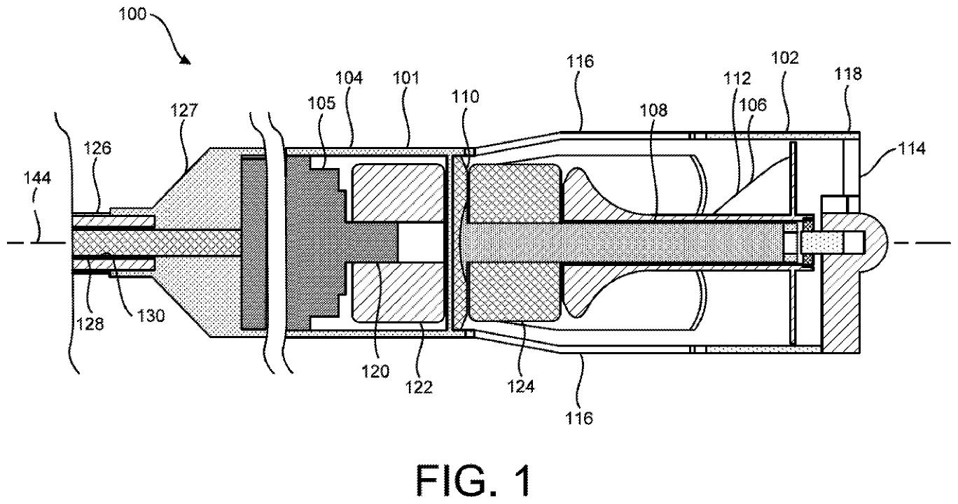

FIG. 1 depicts a partial side sectional view of an illustrative percutaneous circulatory support device 100 (also referred to herein, interchangeably, as a “blood pump”) in accordance with embodiments of the subject matter disclosed herein. The device 100 may form part of a percutaneous circulatory support system, together with a guidewire and an introducer sheath (not shown). More specifically, the guidewire and the introducer sheath may facilitate percutaneously delivering the device 100 to a target location within a patient, such as within the patient's heart. Alternatively, the device 100 may be delivered to a different target location within a patient.

With continued reference to FIG. 1, the device 100 generally includes a housing 101 that includes an impeller housing 102 and a motor housing 104. In some embodiments, the impeller housing 102 and the motor housing 104 may be integrally or monolithically constructed. In other embodiments, the impeller housing 102 and the motor housing 104 may be separate components configured to be removably or permanently coupled. In some embodiments, the blood pump 100 may lack a separate motor housing 104 and the impeller housing 102 may be coupled directly to the motor 105 described below, or the motor housing 104 may be integrally constructed with the motor 105 described below.

The impeller housing 102 carries an impeller assembly 106 therein. The impeller assembly 106 includes an impeller shaft 108 that is rotatably supported by at least one bearing, such as a bearing 110. The impeller assembly 106 also includes an impeller 112 that rotates relative to the impeller housing 102 to drive blood through the device 100. More specifically, the impeller 112 causes blood to flow from a blood inlet 114 formed on the impeller housing 102, through the impeller housing 102, and out of a blood outlet 116 formed on the impeller housing 102. In some embodiments and as illustrated, the impeller shaft 108 and the impeller 112 may be separate components, and in other embodiments the impeller shaft 108 and the impeller 112 may be integrated. In some embodiments and as illustrated, the inlet 114 and/or the outlet 116 may each include multiple apertures. In other embodiments, the inlet 114 and/or the outlet 116 may each include a single aperture. In some embodiments and as illustrated, the inlet 114 may be formed on a distal end portion 118 of the housing 101 and the outlet 116 may be formed on a side portion of the housing 101. In other embodiments, the inlet 114 and/or the outlet 116 may be formed on other portions of the housing 101. In some embodiments, the housing 101 may couple to a distally extending cannula (not shown), and the cannula may receive and deliver blood to the inlet 114.

With continued reference to FIG. 1, the motor housing 104 carries a motor 105, and the motor 105 is configured to rotatably drive the impeller 112 relative to the impeller housing 102. In the illustrated embodiment, the motor 105 rotates a drive shaft 120, which is coupled to a driving magnet 122. Rotation of the driving magnet 122 causes rotation of a driven magnet 124, which is connected to and rotates together with the impeller assembly 106. More specifically, in embodiments incorporating the impeller shaft 108, the impeller shaft 108 and the impeller 112 are configured to rotate with the driven magnet 124. In other embodiments, the motor 105 may couple to the impeller assembly 106 via other components.

In some embodiments, a controller (not shown) may be operably coupled to the motor 105 and configured to control the motor 105. In some embodiments, the controller may be disposed within the motor housing 104. In other embodiments, the controller may be disposed outside of the motor housing 104 (for example, in an independent housing, etc.). In some embodiments, the controller may include multiple components, one or more of which may be disposed within the motor housing 104. According to embodiments, the controller may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller is referred to herein in the singular, the controller may be implemented in multiple instances, distributed across multiple computing devices, instantiated within multiple virtual machines, and/or the like. In other embodiments, the motor 105 may be controlled in other manners.

With continued reference to FIG. 1, the motor housing 104 couples to a catheter 126 on a proximal end portion 127 of housing 101. The catheter 126 may couple to the motor housing 104 in various manners, such as laser welding, soldering, via an adhesive, via polymer reflow, or the like. The catheter 126 extends proximally away from the motor housing 104. The catheter 126 carries a motor cable 128 within a lumen 130, and the motor cable 128 may operably couple the motor 105 to the controller (not shown) and/or an external power source (not shown).

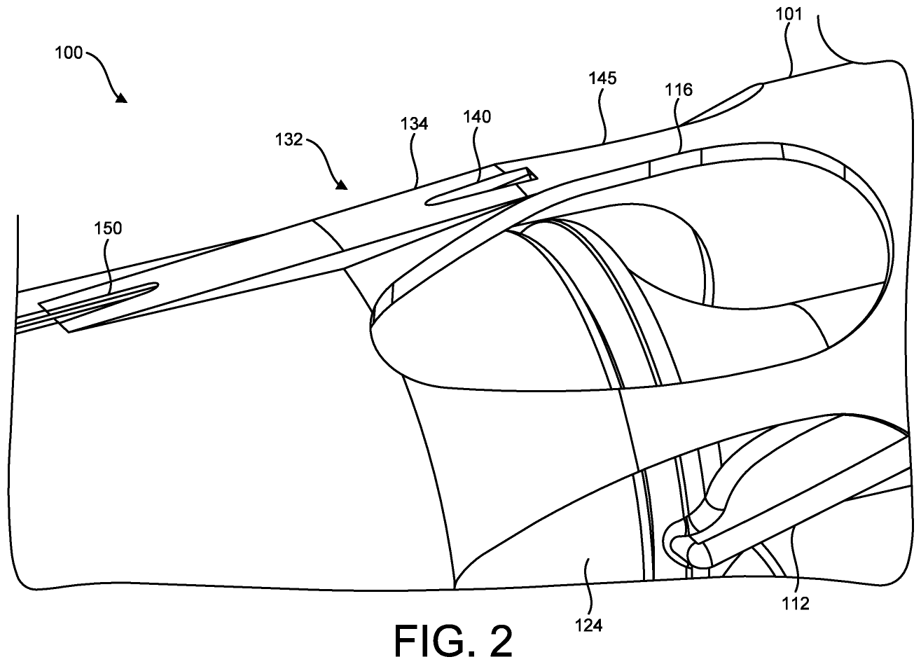

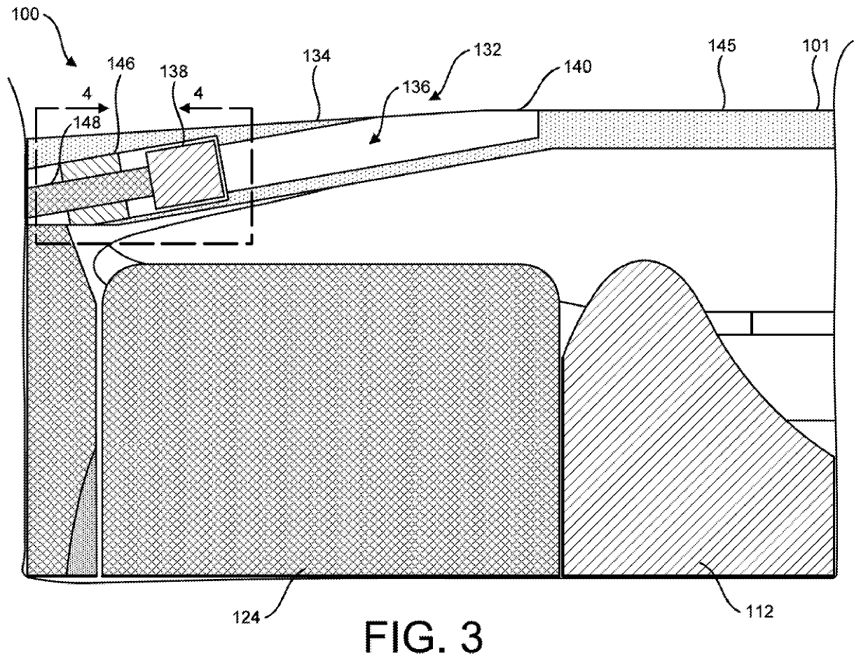

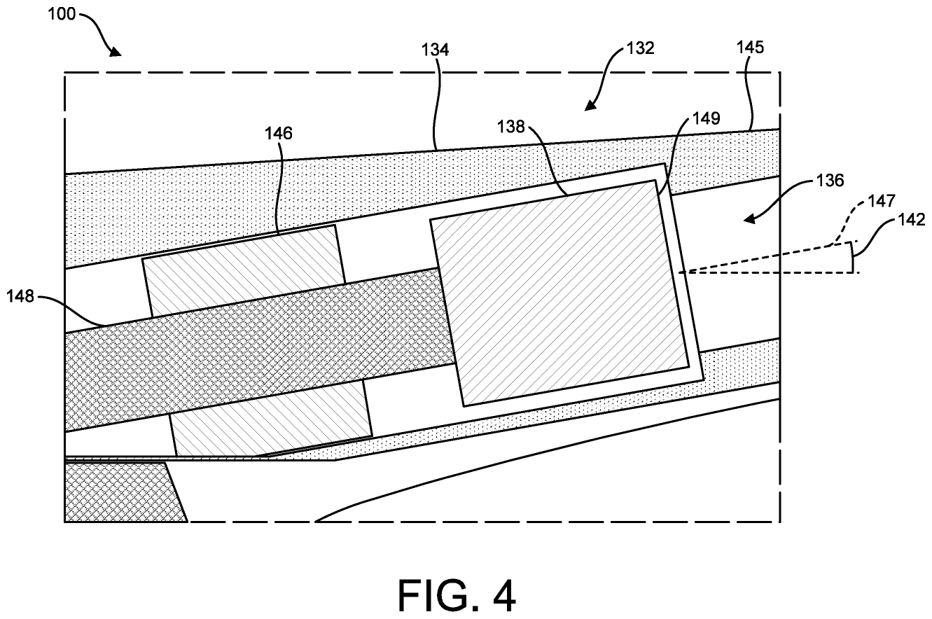

Referring now to FIGS. 2-4, the device 100 further includes a sensor assembly 132 for measuring pressure within the vasculature of a patient, for example, within the aorta. The sensor assembly 132 includes a sensor housing 134 having an internal chamber 136 (FIGS. 3 and 4). A pressure sensor 138 (FIGS. 3 and 4), such as an optical or electrical pressure sensor, is disposed within the internal chamber 136. The sensor housing 134 protects the pressure sensor 138 during deployment of the device 100. The sensor housing 134 also includes a transversely-facing distal aperture 140 (FIGS. 2 and 3; as used herein, the term “transverse” and variations meaning non-parallel to a longitudinal axis of the impeller housing 102) coupled to the internal chamber 136. The aperture 140 permits blood to enter the internal chamber 136, and the aperture 140 thereby permits the pressure sensor 138 to sense the pressure of the blood.

As illustrated most clearly in FIG. 4, the pressure sensor 138 may be disposed at a non-zero angle 142 relative to the longitudinal axis 144 of the impeller housing 102 (FIG. 1). More specifically, a direction 147 perpendicular to the sensing membrane 149 of the sensor 138, at its distal surface, may be disposed at a non-zero angle 142 relative to the longitudinal axis 144 of the impeller housing 102. The disposition of the pressure sensor 138 reduces dynamic pressure-related sensing inaccuracies. The non-zero angle 142 may be, for example, in a range of 7.5 degrees to 90 degrees. In some embodiments, the angle 142 may be a non-zero acute angle in a range of 7.5 degrees to 45 degrees, more specifically in a range of 7.5 degrees to 30 degrees, more specifically in a range of 7.5 degrees to 12.5 degrees, more specifically in a range of 9 degrees to 11 degrees, and even more specifically 10 degrees. As illustrated, the sensor assembly 132, and more specifically the pressure sensor 138 and/or a transversely-facing distal aperture 140 of the internal chamber 136, may be located on, or near, one of the struts 145 between the apertures of the outlet 116. Such a structure facilitates determining if the device 100 inadvertently and improperly moves such that the outlet 116 is located at or near the aortic valve, or within the left ventricle, based on pressure signals that significantly differ from those obtained if the outlet 116 is located in the aorta and apart from the aortic valve.

With general reference again to FIGS. 2-4, the sensor housing 134 may take various forms. For example and as illustrated, the sensor housing 134 may be integrally or monolithically constructed with the impeller housing 102. Alternatively, the sensor housing 134 may be a separately constructed component, such as a tube or ferrule manufactured from, for example, one or more metals, one or more plastics, composites, or the like. The sensor housing 134 may also include a sensor mount 146 within the internal chamber 136. The sensor mount 146 facilitates supporting the pressure sensor 138 apart from the walls of the sensor housing 134 (that is, the sensor mount 146 centers the pressure sensor 138 within the internal chamber 136), which in turn facilitates high-accuracy pressure sensing.

The sensor assembly 132 further includes a sensor cable 148 coupled to the pressure sensor 138. The sensor cable 148 may operably couple the pressure sensor to the controller (not shown). As illustrated, the sensor cable 148 may extend through the sensor mount 146 and support the pressure sensor 138 apart from the walls of the sensor housing 134. The sensor cable 148 extends proximally through a proximally-facing aperture 150 (FIG. 2) of the sensor housing 134 disposed proximally relative to the blood outlet 116. In some embodiments, the sensor cable 148 may additionally extend through a cable lumen (not shown) coupled to or constructed as part of the catheter 126 (FIG. 1).

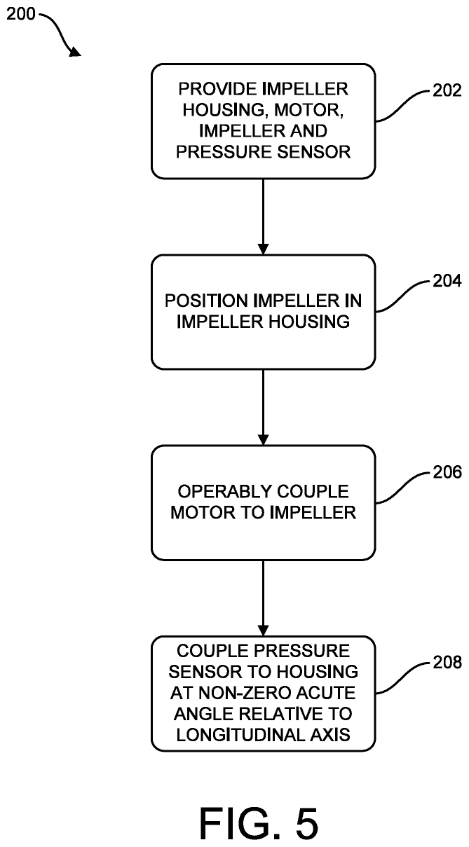

FIG. 5 illustrates a flow diagram of an exemplary method 200 of manufacturing a blood pump, in accordance with embodiments of the subject matter disclosed herein. The method 200 describes features of the blood pump 100, although it is understood that any of the blood pumps contemplated herein could be manufactured in a similar manner. At block 202, the method begins by providing the impeller housing 102, the motor 105, the impeller 112, and the sensor 138. At block 204, the impeller 112 is positioned in the housing 101 such that the impeller 112 is rotatable relative to the housing 101. More specifically, the impeller 112 is coupled to the impeller shaft 108. At block 206, the motor 105 is operably coupled to the impeller 112, for example, via the drive magnet 122 and the driven magnet 124. At block 208, the pressure sensor 138 is coupled to the housing 101 such that the pressure sensor 138 is disposed at the non-zero angle 142 relative to the longitudinal axis 144 of the housing 101. More specifically, the pressure sensor 138 is inserted through the proximally-facing aperture 150 and into the internal chamber 136 of the sensor housing 134.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A percutaneous circulatory support device, comprising:
a housing comprising an inlet, an outlet, a proximal end portion, a distal end portion, and a longitudinal axis extending between the proximal end portion and the distal end portion;
an impeller disposed within the housing, the impeller configured to rotate relative to the housing to cause blood to flow into the inlet, through the housing, and out of the outlet;
a motor operably coupled to the impeller, the motor configured to rotate the impeller relative to the housing; and
a pressure sensor coupled to the housing and disposed at a non-zero angle relative to the longitudinal axis.

2. The percutaneous circulatory support device of claim 1, wherein the housing further comprises an internal chamber and an aperture coupled to the internal chamber, and the pressure sensor is disposed within the internal chamber.

3. The percutaneous circulatory support device of claim 2, wherein the aperture is a transversely-facing aperture.

4. The percutaneous circulatory support device of claim 2, further comprising a sensor mount disposed within the internal chamber and coupled to the pressure sensor.

5. The percutaneous circulatory support device of claim 2, further comprising a sensor cable coupled to the pressure sensor.

6. The percutaneous circulatory support device of claim 5, wherein the aperture is a transversely-facing aperture, the housing further comprises a proximally-facing aperture coupled to the internal chamber, and the sensor cable extends through the proximally-facing aperture.

7. The percutaneous circulatory support device of claim 2, wherein the housing further comprises a plurality of outlet apertures defining the outlet and a plurality of struts disposed between the plurality of outlet apertures, and the pressure sensor is carried by one of the plurality of struts.

8. The percutaneous circulatory support device of claim 2, wherein the pressure sensor comprises one of an optical pressure sensor and an electrical pressure sensor.

9. The percutaneous circulatory support device of claim 1, wherein a direction perpendicular to a sensing membrane of the pressure sensor is disposed at the non-zero angle relative to the longitudinal axis.

10. A percutaneous circulatory support device, comprising:
a housing comprising:
an inlet;
an outlet comprising a plurality of outlet apertures;
a plurality of struts disposed between the plurality of outlet apertures;
an impeller disposed within the housing, the impeller configured to rotate relative to the housing to cause blood to flow into the inlet, through the housing, and out of the outlet;
a motor operably coupled to the impeller, the motor configured to rotate the impeller relative to the housing; and
a pressure sensor carried by one of the plurality of struts.

11. The percutaneous circulatory support device of claim 10, wherein the housing further comprises an internal chamber and an aperture coupled to the internal chamber, and the pressure sensor is disposed within the internal chamber.

12. The percutaneous circulatory support device of claim 11, wherein the aperture is a transversely-facing aperture.

13. The percutaneous circulatory support device of claim 12, wherein the housing further comprises a proximally-facing aperture coupled to the internal chamber.

14. The percutaneous circulatory support device of claim 13, further comprising a sensor cable coupled to the pressure sensor and extending through the proximally-facing aperture.

15. The percutaneous circulatory support device of claim 13, wherein the proximally-facing aperture is disposed proximally relative to the outlet.

16. The percutaneous circulatory support device of claim 10, further comprising a sensor mount disposed within the internal chamber and coupled to the pressure sensor.

17. A method of manufacturing a percutaneous circulatory support device, the method comprising:
positioning an impeller within a housing such that the impeller is rotatable relative to the housing;
operably coupling a motor to the impeller; and
coupling a pressure sensor to the housing such that the pressure sensor is disposed at non-zero angle relative to a longitudinal axis of the housing.

18. The method of claim 17, wherein coupling the pressure sensor to the housing comprises positioning the pressure in an internal chamber of a sensor housing.

19. The method of claim 18, wherein the sensor housing further comprises an aperture coupled to the internal chamber.

20. The method of claim 19, wherein the aperture is a transversely-facing aperture, the sensor housing further comprises a proximally-facing aperture, and coupling the pressure sensor to the housing comprises inserting the pressure sensor through the proximally-facing aperture.
